(19) United States
(12) Patent Application Publication
(10) Pub. No.: US 2024/0331818 A1
(43) Pub. Date: Oct. 3, 2024

(54) MEDICAL INFORMATION STORING SYSTEM, MEDICAL INFORMATION STORING METHOD, AND RECORDING MEDIUM

(71) Applicant: Konica Minolta, Inc., Tokyo (JP)

(72) Inventor: Hiroshi Nakamori, Tokyo (JP)

(73) Assignee: Konica Minolta, Inc., Tokyo (JP)

(21) Appl. No.: 18/621,592

(22) Filed: Mar. 29, 2024

(30) Foreign Application Priority Data
Mar. 30, 2023 (JP) 2023-055603

(51) Int. Cl. G16H 10/60; G16H 30/40

(52) U.S. Cl. CPC G16H 10/60; G16H 30/40

(57) ABSTRACT

A medical information storing system includes: a hardware processor that: stores medical data related to medical care in a first storage provided in a cloud; transfers, from the first storage, the medical data satisfying a designated condition to a second storage provided in the cloud, wherein the second storage requires a longer reading time period than the first storage does; manages a storing state of the medical data; and outputs to a display the storing state, or warning information about restoration of the medical data stored in the second storage and having been referred to.

### CROSS-REFERENCE TO RELATED APPLICATIONS

The entire disclosure of Japanese Patent Application No. 2023-055603 filed on Mar. 30, 2023, including description, claims, drawings and abstract is incorporated herein by reference.

### BACKGROUND

### Technical Field

The present invention relates to a medical information storing system, a medical information storing method, and a recording medium.

### Description Of Related Art

In recent years, a system that stores and manages medical image data in a storage in the cloud, for backing up and archiving the medical image data, has been used. Accordingly, desired medical images can be referred to even from external apparatuses used for remote community healthcare, home medical care and the like, without limitation to those inside of a medical facility.

Storage services in the cloud are variously classified according to the data reading rate and writing rate, etc. For example, services of a high-speed storage that has a high reading rate and writing rate, and a high price, and those of a low-speed storage that has a low reading rate and writing rate, requires restoration, and has a low price, are provided.

JP 2017-76159A describes a technique of managing medical information using a plurality of storages. Specifically, a technique is described that moves medical information between storage devices having different access rates, based on the status of a patient and storage level information.

Although according to JP 2017-76159A the medical information is moved between the storage devices having different access rates, it is assumed that the plurality of storages are arranged in a hospital facility. Thus, a technical issue specific to the cloud in a case of using storages in the cloud, for example, a technical issue that a low-speed storage takes a certain time period to restore files, is not considered.

### SUMMARY

One or more embodiments of the present invention provide a medical information storing system, a medical information storing method, and a recording medium that improve functionalities of the system, especially control technique for storages having different performances, and thus provide improvements in medical information management technology.

According to an aspect of the present invention, a medical information storing system includes a hardware processor that:
stores medical data related to medical care in a first storage provided in a cloud;
transfers, from the first storage, the medical data satisfying a designated condition to a second storage provided in the cloud, wherein the second storage requires a longer reading time period than the first storage does;
manages a storing state of the medical data; and
outputs to a display the storing state, or warning information about restoration of the medical data stored in the second storage and having been referred to.

According to an aspect of the present invention, a medical information storing method causes a hardware processor to perform:
storing medical data related to medical care in a first storage provided in a cloud;
transferring, from the first storage, the medical data satisfying a designated condition to a second storage provided in the cloud, wherein the second storage requires a longer reading time period than the first storage does;
managing a storing state of the medical data; and
outputting to a display the storing state, or warning information about restoration of the medical data stored in the second storage and having been referred to.

According to an aspect of the present invention, a recording medium is a non-transitory computer readable recording medium storing instructions causing a computer to execute:
storing medical data related to medical care in a first storage provided in a cloud;
transferring, from the first storage, the medical data satisfying a designated condition to a second storage provided in the cloud, wherein the second storage requires a longer reading time period than the first storage does;
managing a storing state of the medical data; and
outputting to a display the storing state, or warning information about restoration of the medical data stored in the second storage and having been referred to.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows an example of a schematic configuration of a medical information storing system according to one or more embodiments;

FIG. 2A shows an example of a configuration of a management table on a cloud side according to one or more embodiments;

FIG. 2B shows a list of status codes used in the management table in one or more embodiments;

FIG. 3 shows a block diagram of a medical image management apparatus according to one or more embodiments;

FIG. 4A shows an example of a configuration of a management table on a hospital facility side according to one or more embodiments;

FIG. 4B shows a list of status codes used in the management table in one or more embodiments;

FIG. 5 is a block diagram of an information processing apparatus according to one or more embodiments;

FIG. 6 is a sequence diagram of the medical information storing system in a case of moving image data stored in a standard storage in the cloud to an inexpensive storage according to one or more embodiments;

FIG. 7 is a sequence diagram of the medical information storing system in a case of restoring image data stored in the inexpensive storage in the cloud into the standard storage in response to a reference request by a user in a hospital facility;

FIG. 8 shows an example of warning information displayed on a display of the information processing apparatus according to one or more embodiments;

FIG. 9 is a sequence diagram of the medical information storing system in a case of restoring image data stored in the inexpensive storage in the cloud into the standard storage in response to a reference request by a user out of the hospital facility; and

FIG. 10 is a sequence diagram of the medical information storing system in a case of restoring image data stored in the inexpensive storage in the cloud into the standard storage in response to a reference request by a user out of the hospital facility.

### DETAILED DESCRIPTION

Hereinafter, embodiments of the present invention will be described with reference to the drawings. However, the scope of the invention is not limited to the disclosed embodiments.

### [Configuration Example of Medical Information Storing System 10]

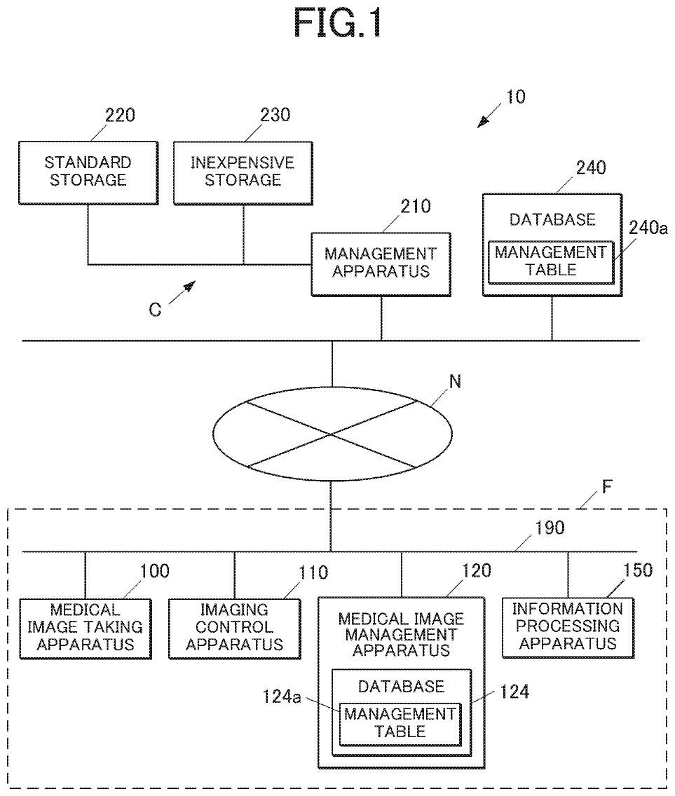

FIG. 1 shows an example of a schematic configuration of a medical information storing system 10 according to one or more embodiments.

As shown in FIG. 1, the medical information storing system 10 includes a medical image taking apparatus 100, an imaging control apparatus 110, a medical image management apparatus 120, an information processing apparatus 150, a management apparatus 210, a standard storage 220, an inexpensive storage 230, and a database 240. The standard storage 220 functions as a first storage. The inexpensive storage 230 is also called a cold storage, and functions as a second storage. The database 240 functions as a second database.

The medical image taking apparatus 100, the imaging control apparatus 110, the medical image management apparatus 120, and the information processing apparatus 150 are provided in a hospital facility F, for example. The medical image taking apparatus 100, the imaging control apparatus 110, the medical image management apparatus 120, and the information processing apparatus 150 are connected to each other by a network 190, such as a local area network (LAN). The network 190 may be wired or wireless one. The management apparatus 210, the standard storage 220, the inexpensive storage 230, and the database 240 are provided in a cloud C. The hospital facility F and the cloud C are connected to each other via a network N. The network N may be, for example, the Internet, a wide area network (WAN), etc. Note that in one or more embodiments, the cloud C means a utilization form of a computer for providing a predetermined service using hardware and software on the network N, such as the Internet.

The medical image taking apparatus (modality) 100 takes a medical image of a patient (subject), and generates image data of the taken medical image of the patient. The medical image may be a still image, or a dynamic image including a plurality of frames. The image data arc examples of medical data related to medical care, and the medical image data. The medical image taking apparatus 100 may be of, for example, computed radiography (CR), digital radiography (DR), computed tomography (CT), magnetic resonance imaging (MRI), ultra sonography (US), nuclear medicine (NM), endoscope(ES), i.e., endoscopy, or the like. The medical image taking apparatus 100 adds attribute information pertaining to the taken medical image, to this medical image. The attribute information includes, for example, a patient ID, a patient name, an age, an examination apparatus, an examination site, and an examination date. The medical image taking apparatus 100 transmits the generated image data of the medical image, and the attribute information added to the image data, to the medical image management apparatus 120.

The imaging control apparatus 110 receives the image data in accordance with the still image or the dynamic image, from the medical image taking apparatus 100. The imaging control apparatus 110 is made up of a computer and the like, and has a function of a console. The imaging control apparatus 110 sets an imaging condition in the medical image taking apparatus 100 and the like, based on order information or the like transmitted from an RIS or the like. The imaging condition includes, for example, a tube voltage [kV], a tube current [mA], an irradiation time period [ms], a radiation dose [mAs], a source-to-image distance (SID) [cm], presence or absence of an additional filter, and a filter type.

The medical image management apparatus 120 stores and manages the image data, the attribute information and the like in accordance with the medical image generated by the medical image taking apparatus 100. For the sake of image data backing up, archiving and the like, the medical image management apparatus 120 stores image data in the standard storage 220 and the inexpensive storage 230 provided in the cloud C. The medical image management apparatus 120 includes a database 124 that associates the image data with the attribute information, a status indicating the storing state of the image data, and the like, and manages them. The medical image management apparatus 120 functions as a picture archiving and communication system (PACS), an image diagnostic workstation or the like.

The information processing apparatus 150 is a computer or the like used by a user, such as an interpretation doctor or an examining doctor, in the hospital facility F. The information processing apparatus 150 reads and displays the image data stored in the medical image management apparatus 120, for example, in response to a user's reference request for a predetermined medical image. Note that the number of information processing apparatuses 150 may be one or more.

The management apparatus 210 stores the image data in at least one of the standard storage 220 and the inexpensive storage 230, and manages the stored image data, based on, for example, control by the medical image management apparatus 120. Specifically, the management apparatus 210 stores, in the standard storage 220, the image data transmitted from the medical image management apparatus 120. The management apparatus 210 moves the image data satisfying a designated condition from the standard storage 220 to the inexpensive storage 230. If the reference request for the predetermined image data has been issued and the image data are stored in the inexpensive storage 230, the management apparatus 210 restores the image data for which the reference request has been issued, from the inexpensive storage 230 into the standard storage 220. Similar to the medical image management apparatus 120 described later, the management apparatus 210 includes a processor, such as a CPU, and a memory.

The standard storage 220 has a higher data reading rate and writing (writing into) rate than the inexpensive storage 230 does. The standard storage 220 may be made up of, for example, a semiconductor memory, such as a solid state drive (SSD). A storage other than an SSD can be adopted. The standard storage 220 is connected to the management apparatus 210 and the inexpensive storage 230 via an interface, such as a bus, for example. The standard storage 220 stores, removes, and applies another action to the image data, based on an instruction by the management apparatus 210.

The inexpensive storage 230 has lower data reading and writing (writing into) rates than the standard storage 220 does, requires restoration, and is more inexpensive than the standard storage 220. The inexpensive storage 230 may be made up of, for example, a hard disk drive (HDD), or made up of an optical disk drive, magnetic tape or the like, or a combination of them. The inexpensive storage 230 is connected to the management apparatus 210 and the standard storage 220 via an interface, such as a bus, for example. The inexpensive storage 230 stores the image data and copies it to the standard storage 220, and applies another action to these data, based on an instruction by the management apparatus 210.

In one or more embodiments, for the sake of backing up and archiving, the image data stored in the medical image management apparatus 120 are stored in the standard storage 220 in the cloud C. If the image data stored in the standard storage 220 satisfy a condition that is designated (hereinafter called a designated condition), the image data are moved to the inexpensive storage 230. Here, the designated condition may be, for example, an examination date of a patient or an examination receipt date. Another condition may be, for example, a lapse of a predetermined period from a latest examination registration of the patient, or a lapse of a predetermined period from hospital visiting of the patient. Still another designated condition may be, for example, death of a patient, or complete recovery of the patient.

If a reference request is issued by the user, the image data stored in the inexpensive storage 230 are moved to the standard storage 220, and restored therein. The image data having been moved to the standard storage 220 and restored therein are downloaded to the information processing apparatus 150 or the like having issued the reference request. There are herein two processing methods of moving the image data from the inexpensive storage 230 to the standard storage 220. The first method temporarily copies the image data from the inexpensive storage 230 to the standard storage 220, and restores the image data. The image data remain in the inexpensive storage 230. On the other hand, the image data restored in the standard storage 220 are removed after a lapse of a designated period set by the user. The second method copies the image data from the inexpensive storage 230 to the standard storage 220. The image data in the inexpensive storage 230 are removed after completion of copying. The standard storage 220 stores the copied image data.

The database 240 includes a management table 240a that manages the image data generated by the medical image taking apparatus 100, in association with, for example, attribute information including examination information and patient information, a status indicating the storing state of the image data, and the like.

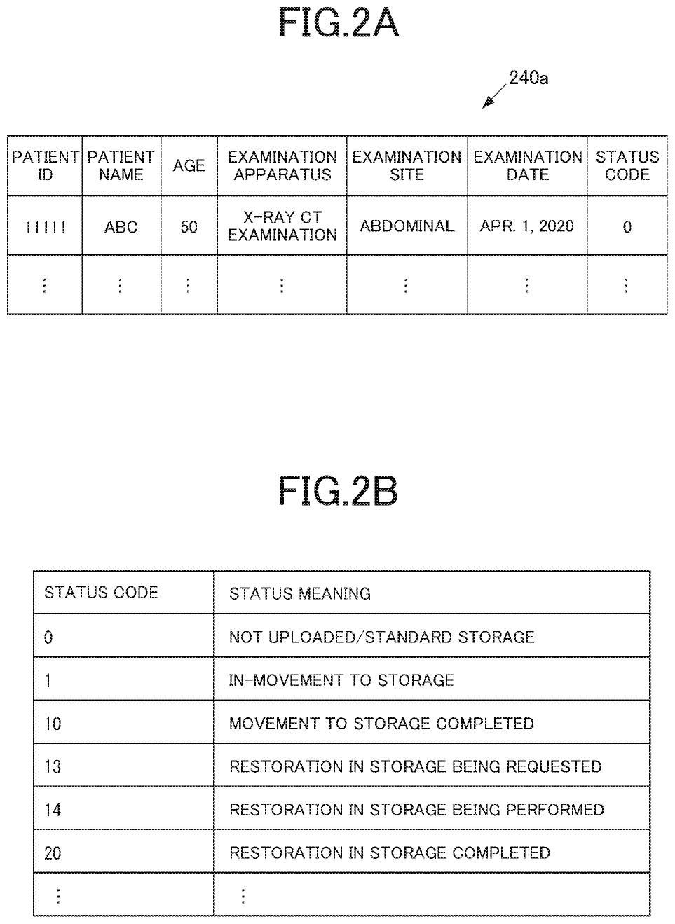

FIG. 2A shows an example of the configuration of the management table 240a. The management table 240a stores, for example, the patient ID, patient name, age, examination apparatus, examination site, examination date, and status code. Here, the status code is information indicating the storing state of the image data in the standard storage 220 and the inexpensive storage 230 in the cloud C. The storing state includes, for example, the storing location of the image data, and the states of movement, restoration and the like of the image data.

FIG. 2B shows a list of status codes used in one or more embodiments. FIG. 2B shows each status code and the meaning of the status code in association with each other. A controller 121 of the medical image management apparatus 120 and the like updates the status code in a management table 124a of the database 124, in accordance with the storing states of the image data in the standard storage 220 and the inexpensive storage 230. In the database 240 on the cloud C side, for example, “0, 1, 10, 13, 14, 20” and the like are used as status codes.

### [Configuration Example of Medical Image Management Apparatus 120]

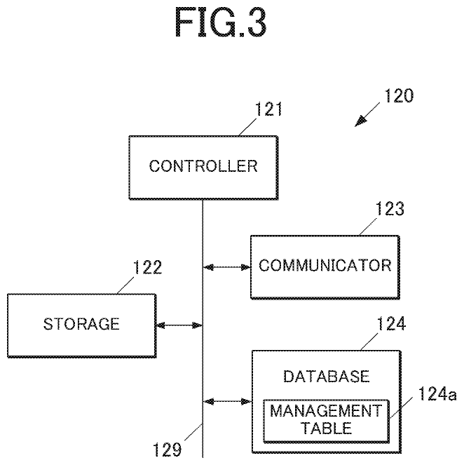

FIG. 3 is a block diagram of the medical image management apparatus 120.

As shown in FIG. 3, the medical image management apparatus 120 includes the controller 121 (i.e., a hardware processor), a storage 122, a communication interface 123, and the database 124. The database 124 functions as a first database. The controller 121, the storage 122, the communication interface 123, and the database 124 are connected to each other via a bus 129. Although not described with reference to FIG. 3, a display and an operation receiver may be provided for the medical image management apparatus 120.

The controller 121 includes: at least a processor, such as a central processing unit (CPU); and a memory, such as a random access memory (RAM). The CPU of the controller 121 reads instructions such as system programs and various processing programs stored in the storage 122, loads the instructions into the RAM, and executes various processes according to the loaded instructions.

In one or more embodiments, the controller 121 achieves the following processes by executing the instructions. The controller 121 functions as a storing device and a storing step that cause the standard storage 220 provided in the cloud C to store the image data. The controller 121 functions as a moving device and a moving step that move the image data satisfying the designated condition, from the standard storage 220 to the inexpensive storage 230 that is provided in the cloud C and requires a longer reading time period than the standard storage 220 does. The controller 121 functions as a management device and a management step that mange the status indicating the storing state of the image data. The controller 121 functions as an output device and an output step that output the status of the image data to a display 154 of the information processing apparatus 150, or the like. The controller 121 also functions as an output device and an output step that output warning information related to restoration of the image data stored in the inexpensive storage 230 and having been referred to. Note that at least one function among the storing device, moving device, management device, and output device described above may be executed through cooperation of other apparatuses, for example, the management apparatus 210, and after-mentioned web servers 160 and 260, information processing apparatus and the like.

The storage 122 includes at least one of a semiconductor memory, a hard disk drive (HDD), an optical disk storage device and the like. The storage 122 stores various instructions such as processing programs, and parameters, files and the like required to execute the instructions. The storage 122 stores image data, such as of medical images, generated by the medical image taking apparatus 100 or the like.

The communication interface 123 includes a communication module. The communication interface 123 transmits and receives various signals and various data to and from external apparatuses wiredly or wirelessly connected via the communication network N. The external apparatuses include the medical image taking apparatus 100, the imaging control apparatus 110, the information processing apparatus 150, and the management apparatus 210.

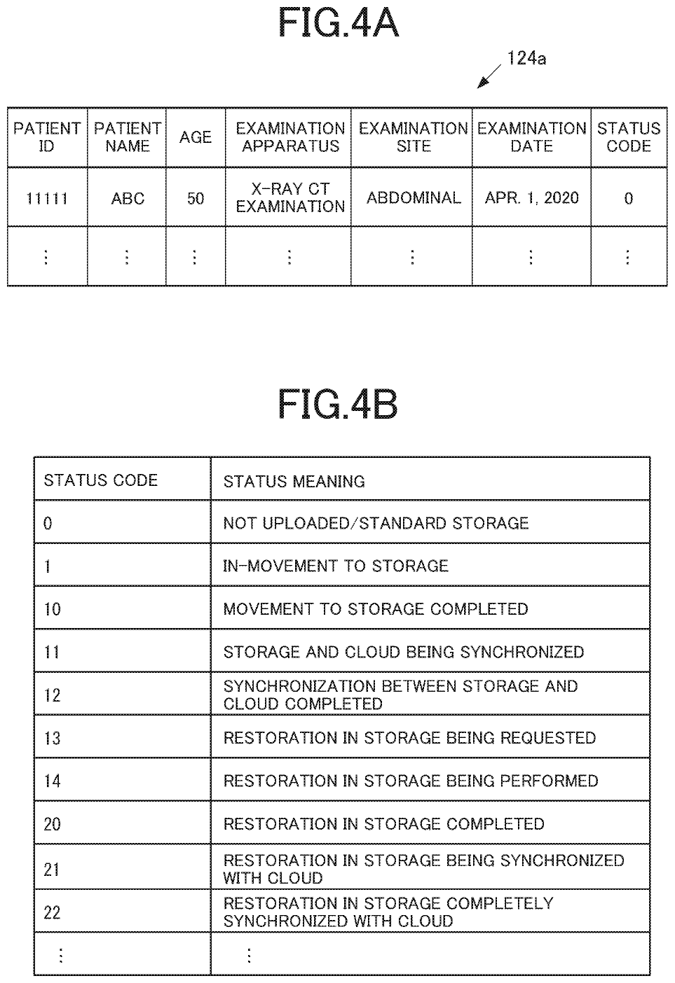

The database 124 includes a management table 124a that manages the image data generated by the medical image taking apparatus 100, in association with attribute information, a status indicating the storing state of the image data, and the like. FIG. 4A indicates an example of the configuration of the management table 124a. The management table 124a stores, for example, the patient ID, patient name, age, examination apparatus, examination site, examination date, and status code. Here, as described above, the status code is information indicating the storing state of each piece of image data in the standard storage 220 and the inexpensive storage 230 in the cloud C. The storing state includes, for example, the storing location of the image data, and the states of movement, restoration and the like of the image data.

FIG. 4B shows a list of status codes used in one or more embodiments. FIG. 4B shows each status code and the meaning of the status code in association with each other. The controller 121 of the medical image management apparatus 120 updates the status code in a management table 124a of the database 124, in accordance with the storing states of the image data in the standard storage 220 and the inexpensive storage 230. In the database 124 on the hospital facility F side, for example, “0, 1, 10, 11, 12, 13, 14, 20, 21, 22” and the like are used as status codes.

### [Configuration Example of Information Processing Apparatus 150]

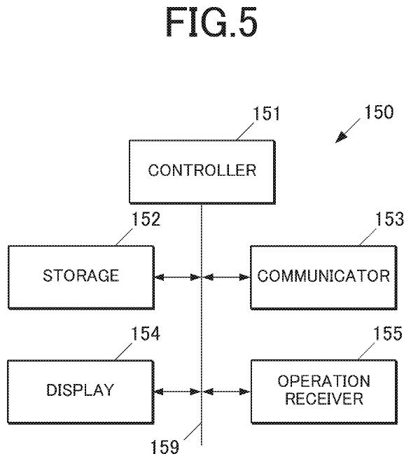

FIG. 5 is a block diagram of the information processing apparatus 150.

As shown in FIG. 5, the information processing apparatus 150 includes a controller 151, a storage 152, a communication interface 153, a display 154, and an operation receiver 155. The controller 151, the storage 152, the communication interface 153, the display 154, and the operation receiver 155 are connected to each other via a bus 159. Note that the display 154 functions as a display device.

The controller 151 includes at least a processor, such as a CPU, a memory, such as a RAM. The CPU of the controller 151 reads instructions such as system programs and various processing programs stored in the storage 152, loads the instructions into the RAM, and executes various processes according to the loaded instructions.

The storage 152 includes at least one of a semiconductor memory, a hard disk drive (HDD), an optical disk storage device and the like. The storage 152 stores various instructions such as processing programs, and parameters, files and the like required to execute the instructions.

The communication interface 153 includes a communication module. The communication interface 153 transmits and receives various signals and various data to and from external apparatuses wiredly or wirelessly connected via the communication network N. The external apparatuses include the medical image taking apparatus 100, the imaging control apparatus 110, the medical image management apparatus 120, and the management apparatus 210.

The display 154 may be a display, such as a liquid crystal display, an organic electro luminescence (EL) display, or a cathode ray tube (CRT). The display 154 displays the medical image taken by the medical image taking apparatus 100, a work list in accordance with the management table 124a for issuing a reference request, such as for restoring the image data, and the like, based on display output control by the controller 151. As described above, the display 154 may be combined with a touch panel that is the operation receiver 155.

The operation receiver 155 includes, for example, a keyboard that includes cursor keys, alphanumeric input keys, and various functional keys, and a pointing device, such as a mouse. The operation receiver 155 accepts various instructions input by the user through an operation on the keyboard and the like, and outputs operation signals in response to the accepted instructions to the controller 151. If the operation receiver 155 has a function of a touch panel, the operation receiver 155 accepts an instruction in accordance with the position of a touch operation by a finger or the like of the user.

### [Operation Example of Medical Information Storing System 10]

### (First Sequence)

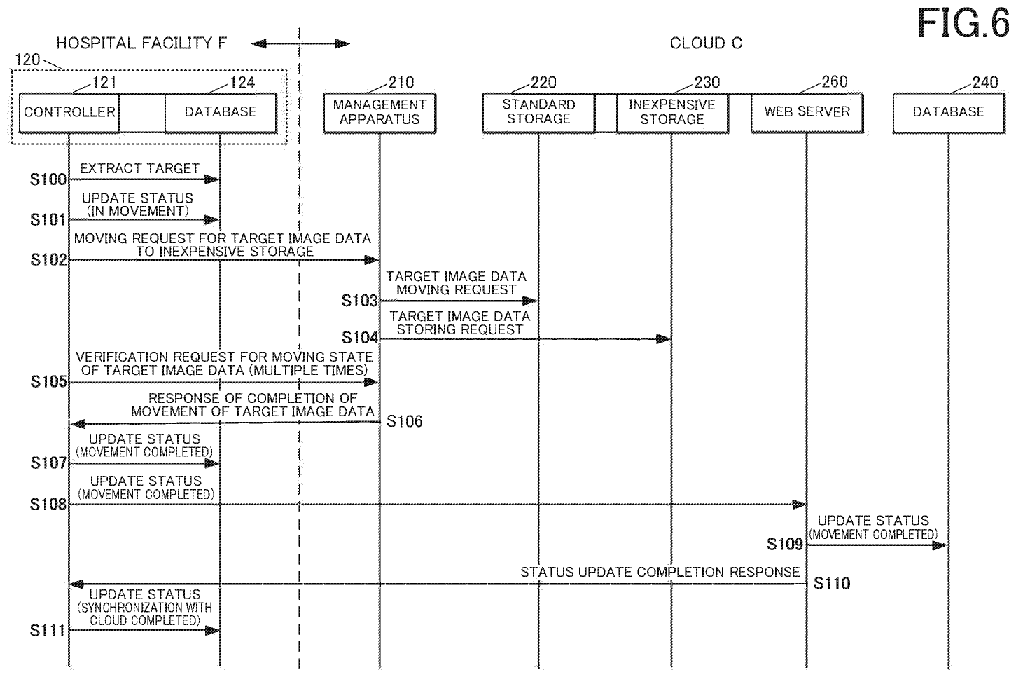

Next, a flow in a case of moving the image data stored in the standard storage 220 to the inexpensive storage 230 is described. FIG. 6 is a sequence diagram of the medical information storing system 10 in the case of moving the image data stored in the standard storage 220 to the inexpensive storage 230 according to one or more embodiments.

First, for the sake of backing up and archiving, the controller 121 of the medical image management apparatus 120 stores the image data of the medical image generated by the medical image taking apparatus 100, in the standard storage 220 in the cloud C, via the network N. The controller 121 functions as a storing device.

The controller 121 of the medical image management apparatus 120 verifies whether image data satisfying a preset designated condition is stored in the standard storage 220, with reference to the management table 124a of the database 124. The controller 121 extracts coincidence information about image data coinciding with the designated condition, from the management table 124a (Step S100).

If there are image data coinciding with the designated condition based on the coincidence information, the controller 121 outputs, to the database 124, an update instruction for updating the status in the database 124 about image data as a target (hereinafter called target image data) (Step S101). The number of pieces of target image data may be one or more. The update instruction includes a status code “1: IN-MOVEMENT TO STORAGE” indicating that the target image data stored in the standard storage 220 are being moved to the inexpensive storage 230. Upon receipt of the update instruction from the controller 121, the database 124 updates the status code of the management table 124a from “0: STANDARD STORAGE” to “1: IN-MOVEMENT TO STORAGE”.

The controller 121 transmits a moving instruction for moving the target image data stored in the standard storage 220 to the inexpensive storage 230, to the management apparatus 210 via the network N (Step S102). The controller 121 functions as a moving device. Here, an authentication key issuing service may be used as a condition for accessing the management apparatus 210 or the like in the cloud C.

Upon receipt of the moving instruction from the medical image management apparatus 120, the management apparatus 210 outputs, to the standard storage 220, the moving instruction for moving the target image data to the inexpensive storage 230 (Step S103). Upon receipt of the moving instruction from the management apparatus 210, the standard storage 220 reads the target image data, and moves the data to the inexpensive storage 230.

Likewise, the management apparatus 210 outputs, to the inexpensive storage 230, a storing instruction for storing the target image data read from the standard storage 220 (Step S104). Upon receipt of the storing instruction from the management apparatus 210, the inexpensive storage 230 stores the target image data read from the standard storage 220.

The controller 121 of the medical image management apparatus 120 transmits a verification instruction for verifying the moving state of the target image data to the management apparatus 210 via the network N (Step S105). If the management apparatus 210 receives the verification instruction from the medical image management apparatus 120, the management apparatus 210 verifies whether the target image data have been moved from the standard storage 220 to the inexpensive storage 230 or is being moved. If the target image data have already been moved from the standard storage 220 to the inexpensive storage 230, the management apparatus 210 transmits a response of completion of movement of the target image data to the medical image management apparatus 120 via the network N (Step S106).

Upon receipt of the response of completion of movement of the target image data from the management apparatus 210, the controller 121 outputs, to the database 124, the update instruction for updating the status of the target image data in the database 124 (Step S107). The controller 121 functions as a management device. The update instruction includes a status code “10: MOVEMENT TO STORAGE COMPLETED” indicating that the movement of the target image data from the standard storage 220 to the inexpensive storage 230 has been completed. Upon receipt of the update instruction from the controller 121, the database 124 updates the status code in the management table 240a from “1: IN-MOVEMENT TO STORAGE” to “10: MOVEMENT TO STORAGE COMPLETED”.

After update of the status in the database 124 in the hospital facility F is completed, the controller 121 transmits an update instruction for updating the status of the target image data in the database 240 in the cloud C, to the web server 260 via the network N (Step S108). The update instruction includes a status code “10: MOVEMENT TO STORAGE COMPLETED” indicating that the movement of the target image data from the standard storage 220 to the inexpensive storage 230 has been completed.

Upon receipt of the update instruction from the medical image management apparatus 120, the web server 260 outputs, to the database 240, the update instruction for updating the status of the target image data in the database 240 in the cloud C (Step S109). Upon receipt of the update instruction from the web server 260, the database 240 updates the status code of the management table 240a from “0: STANDARD STORAGE” to “10: MOVEMENT TO STORAGE COMPLETED”. The status in the management table 240a in the database 240 in the cloud C is updated at this stage, and is synchronized with the status of the target image data in the database 124 in the hospital facility F.

If the update of the status of the target image data in the database 240 is completed, the web server 260 transmits an update completion response to the medical image management apparatus 120 via the network N (Step S110).

Upon receipt of the update completion response from the web server 260, the controller 121 of the medical image management apparatus 120 updates the status code of the target image data in the management table 124a to “22: SYNCHRONIZATION BETWEEN STORAGE AND CLOUD COMPLETED” (Step S111).

### (Second Sequence)

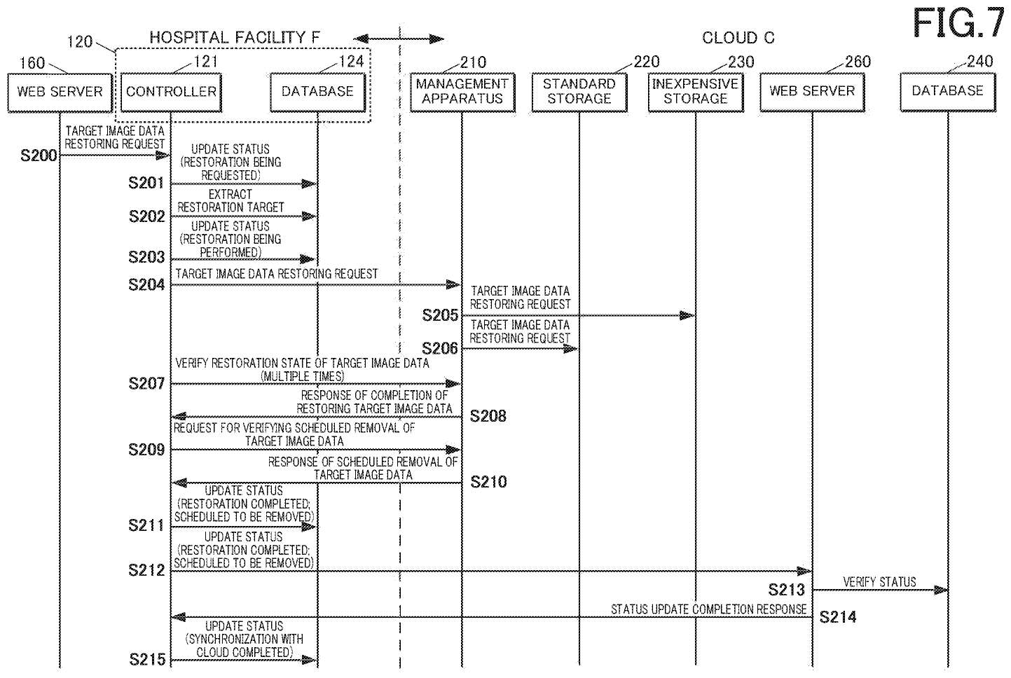

Next, an example of a flow of restoring, in the standard storage 220, the image data stored in the inexpensive storage 230 in the cloud C in the first sequence described above, in response to a reference request by the user in the hospital facility F is described. FIG. 7 is a sequence diagram of the medical information storing system 10 in a case of restoring the image data stored in the inexpensive storage 230 in the cloud C into the standard storage 220 in response to a reference request by the user in the hospital facility F. Note that according to the second sequence, a web server 160 that can accept a reference request for image data, and the like is provided in the hospital facility F.

Upon receipt of a reference request for predetermined image data from the information processing apparatus 150 in the hospital facility F, the web server 160 transmits a restoration request instruction for the target image data, to the controller 121 of the medical image management apparatus 120 (Step S200). For example, the web server 160 causes the display 154 of the information processing apparatus 150 to display a work list, based on display control by the controller 121 of the medical image management apparatus 120. The work list is a list that visually lists attribute information including examination information about the image data and patient information, the status indicating the storing state of the image data, and the like, and is created based on, for example, the management table 124a. The user can verify and select the image data that they intend to refer to from the work list. Note that in one or more embodiments, the controller 121 of the medical image management apparatus 120, and the web server 160 function as output devices.

Here, if the status code of the target image data is “0” or “11” shown in FIG. 4B, and the restoration request date is before a scheduled restoration removal date, the target image data are stored in the standard storage 220. In this case, the target image data are not required to be restored. Accordingly, the user can download the target image data from the standard storage 220 by inputting a reference request for the target image data on the work list.

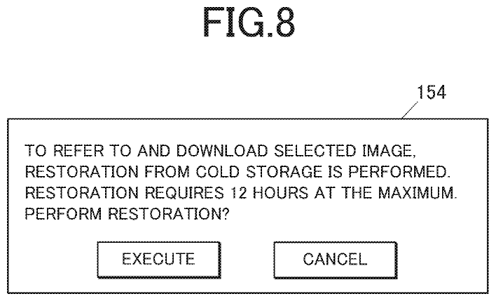

If the status code of the target image data is “1” or the like shown in FIG. 4B, the target image data are stored in the inexpensive storage 230. In this case, a mark indicating presence in the inexpensive storage 230 may be displayed at the target image data on the work list. Restoration of the image data stored in the inexpensive storage 230 into the standard storage 220 requires a considerable time period. Accordingly, the medical image management apparatus 120 may display warning indicating that restoration requires a considerable time period, for the user. FIG. 8 shows an example of warning displayed on the display 154 of the information processing apparatus 150. As shown in FIG. 8, the controller 121 of the medical image management apparatus 120 causes the display 154 to display a message (warning information) indicating that a certain time period is required to restore the target image data, and a button for allowing the user to select whether to download the data (selection information). Here, the controller 121 functions as an output device. For example, if an execution button is selected, a reference request for the target image data is transmitted from the web server 260 to the medical image management apparatus 120. For example, if the execution button is selected, the medical image management apparatus 120 or the web server 260 may display a scheduled restoration time of the target image data to be restored in the standard storage 220, on the display 154 of the information processing apparatus 150. Accordingly, the user can grasp the restoration time period of the target image data, which can facilitate improvement in working efficiency.

Upon receipt of the restoration request for the target image data from the web server 160, the controller 121 of the medical image management apparatus 120 outputs an update instruction for updating the status in the database 124 to the database 124 (Step S201). The update instruction includes a status code “13: RESTORATION IN STORAGE BEING REQUESTED” indicating presence of the request for restoring the target image data stored in the inexpensive storage 230 into the standard storage 220. Upon receipt of the restoration request instruction from the web server 160, the database 124 updates the status code in the management table 124a to “13: RESTORATION IN STORAGE BEING REQUESTED”.

The controller 121 issues a verification request (polling) to the database 124, and extracts, from the management table 124a, restoration target information about the target image data for which the restoration request has been issued (Step S202). The restoration target information includes, for example, patient information, examination information, and status codes.

Based on the extracted restoration target information, the controller 121 outputs, to the database 124, an update instruction for updating the status of the target image data in the database 124 (Step S203). The update instruction includes a status code “14: RESTORATION IN STORAGE BEING PERFORMED” indicating that the target image data stored in the inexpensive storage 230 are being restored into the standard storage 220. Upon receipt of the update instruction from the controller 121, the database 124 updates the status code of the target image data from “13: RESTORATION IN STORAGE BEING REQUESTED” to “14: RESTORATION IN STORAGE BEING PERFORMED”.

The controller 121 transmits a restoration request instruction for restoring the target image data from the inexpensive storage 230 into the standard storage 220, to the management apparatus 210 via the network N (Step S204). The controller 121 functions as a management device. Here, an authentication key issuing service may be used for a condition for accessing the management apparatus 210 or the like in the cloud C.

Upon receipt of the restoration request instruction from the medical image management apparatus 120, the management apparatus 210 outputs, to the inexpensive storage 230, a restoration instruction for moving the target image data to the standard storage 220 (Step S205). Based on the restoration instruction from the management apparatus 210, the inexpensive storage 230 reads the target image data, and moves the data to the standard storage 220.

Likewise, the management apparatus 210 outputs a restoration instruction for temporarily storing the target image data, to the standard storage 220 (Step S206). Based on the restoration instruction from the management apparatus 210, the standard storage 220 stores the target image data read from the inexpensive storage 230. In one or more embodiments, after copying and storing of the target image data to and into the standard storage 220 is completed, the management apparatus 210 can automatically output the restored target image data to the information processing apparatus 150 having issued the reference request. The management apparatus 210 functions as an output device. For example, the management apparatus 210 may remove the target image data stored in the standard storage 220 in accordance with a period set by the user. In this case, the management apparatus 210 can display a scheduled removal time at which the target image data are removed from the standard storage 220, on the display 154 of the information processing apparatus 150 via the medical image management apparatus 120, the web server 160, etc. Accordingly, the user can confirm when the target image data restored in the standard storage 220 are removed.

For example, in a stage of restoring the image data from the inexpensive storage 230 into the standard storage 220, a request for updating the work list is sometimes transmitted from a terminal of another user to the web server or the medical image management apparatus 120. In this case, the web server 160 or the like causes a display of a terminal of another user to display the latest work list, in accordance with the management table 124a, from the database 124. In this stage, “14: RESTORATION IN STORAGE BEING PERFORMED” is displayed as the status of the target image data on the work list.

The controller 121 of the medical image management apparatus 120 transmits a verification instruction for verifying the restoration state of the target image data to the management apparatus 210 via the network N (Step S207). The verification instruction may be issued once, or multiple times in a predetermined period. Upon receipt of the verification instruction from the medical image management apparatus 120, the management apparatus 210 verifies whether restoration of the target image data from the inexpensive storage 230 to the standard storage 220 has been completed, or is being performed. If the target image data have been restored, the management apparatus 210 transmits a response of completion of restoring the target image data, to the medical image management apparatus 120 via the network N (Step S208).

The controller 121 of the medical image management apparatus 120 transmits a verification instruction for verifying scheduled removal of the target image data, to the management apparatus 210 via the network N (Step S209). If the management apparatus 210 receives the verification instruction from the medical image management apparatus 120, the management apparatus 210 transmits a response including information about scheduled removal of the target image data, to the medical image management apparatus 120 via the network N (Step S210).

Upon receipt of the response of scheduled removal of the target image data from the management apparatus 210, the controller 121 outputs, to the database 124, an update instruction for updating the status of the target image data in the database 124 (Step S211). The controller 121 functions as a management device, and an output device. The update instruction includes a status code “20: RESTORATION IN STORAGE COMPLETED” indicating that the restoration of the target image data from the inexpensive storage 230 into the standard storage 220 has been completed. The update instruction includes a status code “22: REMOVAL SCHEDULED” indicating a period or the like after which the target image data will be removed from the standard storage 220. Upon receipt of the update instruction from the controller 121, the database 124 updates the status code of the target image data from “14: RESTORATION IN STORAGE BEING PERFORMED” to “20: RESTORATION IN STORAGE COMPLETED”. “22: REMOVAL SCHEDULED” is added as a status code.

After update of the database 124 in the hospital facility F is completed, the controller 121 transmits an update instruction for updating the status of the target image data in the database 240 in the cloud C, to the web server 260 via the network N (Step S212). The update instruction includes the status code “20: RESTORATION IN STORAGE COMPLETED” indicating that the restoration of the target image data has been completed.

Upon receipt of the update instruction from the medical image management apparatus 120, the web server 260 outputs, to the database 240, the update instruction for updating the status of the target image data in the database 240 in the cloud C (Step S213). Upon receipt of the update instruction from the web server 260, the database 240 updates the status code of the management table 240a from “0: STANDARD STORAGE” to “20: RESTORATION IN STORAGE COMPLETED”. Accordingly, the status of the target image data in the database 240 in the cloud C is synchronized with the status of the target image data of the database 124 in the hospital facility F.

If the update of the status in the database 240 is completed, the web server 260 transmits a status update completion response to the medical image management apparatus 120 via the network N (Step S214).

Upon receipt of the update completion response from the web server 260, the controller 121 of the medical image management apparatus 120 updates the status code of the management table 124a to “22: SYNCHRONIZATION BETWEEN STORAGE AND CLOUD COMPLETED” (Step S215).

### (Third Sequence)

Next, an example of a flow of restoring, into the standard storage 220, the image data stored in the inexpensive storage 230 in the cloud C in the first sequence described above, in response to a reference request by an external user out of the hospital facility F is described. In this example, a case of synchronizing the database 240 in the cloud C with the database 124 in the hospital facility F is described.

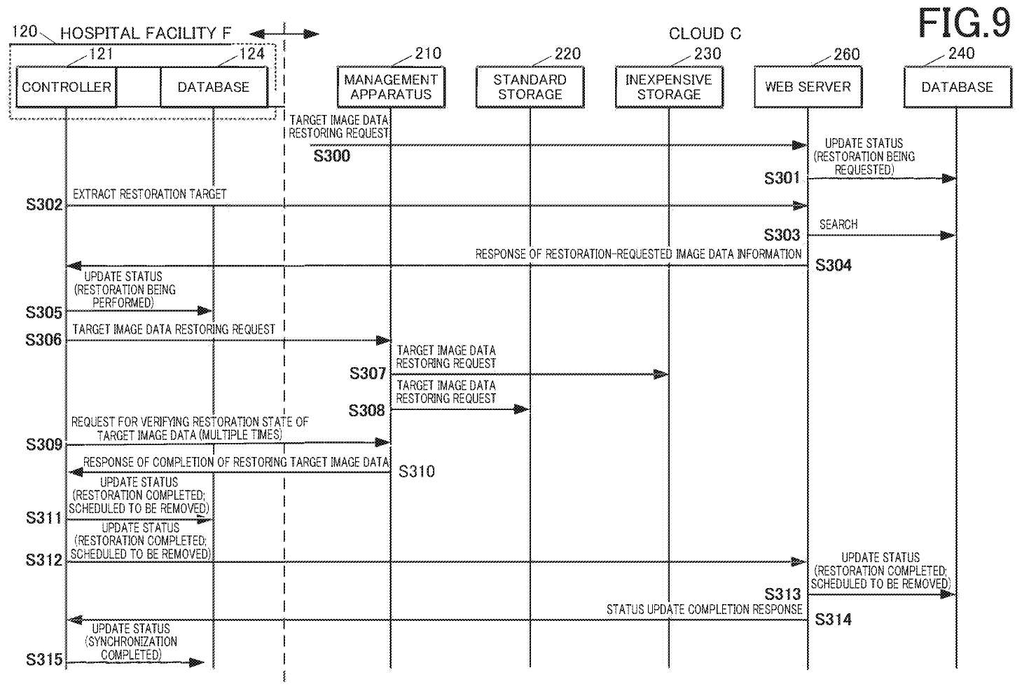

FIG. 9 is a sequence diagram of the medical information storing system 10 in a case of restoring the image data stored in the inexpensive storage 230 in the cloud C into the standard storage 220 in response to a reference request by the external user out of the hospital facility F. Note that according to a third sequence, the web server 260 that manages the database 240 and applies another action is provided in the cloud C. The information processing apparatus provided out of the hospital facility F may have the same components and functions as those of the information processing apparatus 150 shown in FIG. 5, for example.

The web server 260 accepts a reference request for a predetermined target image data, from an external information processing apparatus out of the hospital facility F (Step S300). The user can confirm and select the image data that they intend to refer to from the work list displayed on the display. In this case, as described above, to restore the target image data stored in the inexpensive storage 230 into the standard storage 220, the warning information indicating that a considerable time period is required for restoration, and the selection information for selecting whether to download or not, may be displayed for the user, as shown in FIG. 8.

Upon receipt of the restoration request for the target image data from the external information processing apparatus out of the hospital facility F, the web server 260 outputs, to the database 240, the update instruction for updating the status in the database 240 in the cloud C (Step S301). The web server 260 functions as a management device. The update instruction includes a status code “13: RESTORATION IN STORAGE BEING REQUESTED” indicating that the target image data stored in the inexpensive storage 230 are to be restored into the standard storage 220. Upon receipt of the update instruction from the controller 121, the database 240 updates the status code in the management table 240a to “13: RESTORATION IN STORAGE BEING REQUESTED”.

The controller 121 of the medical image management apparatus 120 in the hospital facility F transmits a verification instruction for verifying (polling) whether a restoration request for image data in the database 240 has been made, to the web server 260 in the cloud C (Step S302).

Upon receipt of the verification instruction from the medical image management apparatus 120, the web server 260 searches the management table 240a in the database 240 (Step S303). The web server 260 extracts restoration target information, such as a status, about the target image data for which the restoration request has been issued (Step S303).

Based on the verification instruction for the database 240, the web server 260 transmits a response including the restoration target information extracted from the database 240, to the medical image management apparatus 120 via the network N (Step S304).

Upon receipt of the restoration target information from the web server 260, the controller 121 outputs, to the database 124, an update instruction for updating the status of the target image data in the database 124 in the hospital facility F (Step S305). Here, the controller 121 functions as a management device. The update instruction includes a status code “14: RESTORATION IN STORAGE BEING PERFORMED” indicating that the target image data stored in the inexpensive storage 230 are being restored into the standard storage 220. Upon receipt of the update instruction from the controller 121, the database 124 updates the status code in the management table 124a from “13: RESTORATION IN STORAGE BEING REQUESTED” to “14: RESTORATION IN STORAGE BEING PERFORMED”.

The controller 121 transmits a restoration request instruction for restoring the target image data from the inexpensive storage 230 into the standard storage 220, to the management apparatus 210 via the network N (Step S306). Here, an authentication key issuing service may be used as a condition for accessing the management apparatus 210 or the like in the cloud C.

Upon receipt of the restoration request instruction from the medical image management apparatus 120, the management apparatus 210 outputs, to the inexpensive storage 230, a restoration instruction for moving the target image data to the standard storage 220 (Step S307). Upon receipt of the restoration instruction from the management apparatus 210, the inexpensive storage 230 reads the target image data, and moves the data to the standard storage 220.

Likewise, the management apparatus 210 outputs a restoration instruction for restoring the target image data, to the standard storage 220 (Step S308). Upon receipt of the restoration instruction from the management apparatus 210, the standard storage 220 stores the target image data read from the inexpensive storage 230. In one or more embodiments, after copying and storing of the target image data to and into the standard storage 220 are completed, the management apparatus 210 may automatically output the restored target image data to the information processing apparatus 150 having issued the reference request. For example, the management apparatus 210 may remove the target image data stored in the standard storage 220 in accordance with a period set by the user. In this case, the scheduled removal time at which the target image data are removed from the standard storage 220 may be displayed on the display 154 of the information processing apparatus 150.

The controller 121 of the medical image management apparatus 120 transmits a verification instruction for verifying the restoration state of the target image data to the management apparatus 210 via the network N (Step S309). The verification instruction is issued once and then finished in some cases, and is issued multiple times and then finished in other cases. In this case, as shown in FIG. 7, the verification instruction described above may include a verification instruction for confirming scheduled removal of the target image data.

Upon receipt of the verification instruction from the medical image management apparatus 120, the management apparatus 210 verifies whether restoration of the target image data from the inexpensive storage 230 to the standard storage 220 has been completed, or is being performed. If the target image data have been restored, the management apparatus 210 transmits a response of completion of restoring the target image data, to the medical image management apparatus 120 via the network N (Step S310).

Upon receipt of the response of completion of restoring the target image data from the management apparatus 210, the controller 121 outputs, to the database 124, the update instruction for updating the status of the target image data in the database 124 (Step S311). The controller 121 functions as a management device, and an output device. The update instruction includes a status code “20: RESTORATION IN STORAGE COMPLETED” indicating that the restoration of the target image data from the inexpensive storage 230 into the standard storage 220 has been completed. Upon receipt of the update instruction from the controller 121, the database 124 updates the status code in the management table 124a from “14: RESTORATION IN STORAGE BEING PERFORMED” to “20: RESTORATION IN STORAGE COMPLETED”. In this case, a status code about scheduled removal of the target image data may be added.

If the controller 121 receives a response of completion of restoring the target image data from the management apparatus 210, the controller 121 transmits an update instruction for updating the status of the target image data in the database 240 in the cloud C, to the web server 260 via the network N (Step S312). The update instruction includes the status code “20: RESTORATION IN STORAGE COMPLETED” indicating that the restoration of the target image data has been completed.

Upon receipt of the update instruction from the medical image management apparatus 120, the web server 260 transmits, to the database 240, the update instruction for updating the status of the target image data in the database 240 in the cloud C (Step S313). Upon receipt of the update instruction from the web server 260, the database 240 updates the status code of the target image data from “13: RESTORATION IN STORAGE BEING REQUESTED” to “20: RESTORATION IN STORAGE COMPLETED” or the like. At this stage, the status of the target image data in the database 240 in the cloud C is synchronized with the status of the target image data of the database 124 in the hospital facility F.

If the update of the status in the database 240 in the cloud C is completed, the web server 260 transmits a status update completion response to the medical image management apparatus 120 via the network N (Step S314).

Upon receipt of the update completion response from the web server 260, the controller 121 of the medical image management apparatus 120 updates the status code of the management table 124a to “22: SYNCHRONIZATION BETWEEN STORAGE AND CLOUD COMPLETED” (Step S315).

### (Fourth Sequence)

Next, an example of a flow of restoring, into the standard storage 220, the image data stored in the inexpensive storage 230 in the cloud C in the first sequence described above, in response to a reference request by an external user out of the hospital facility F is described. In this example, a case of not synchronizing the database 240 in the cloud C with the database 124 in the hospital facility F is described.

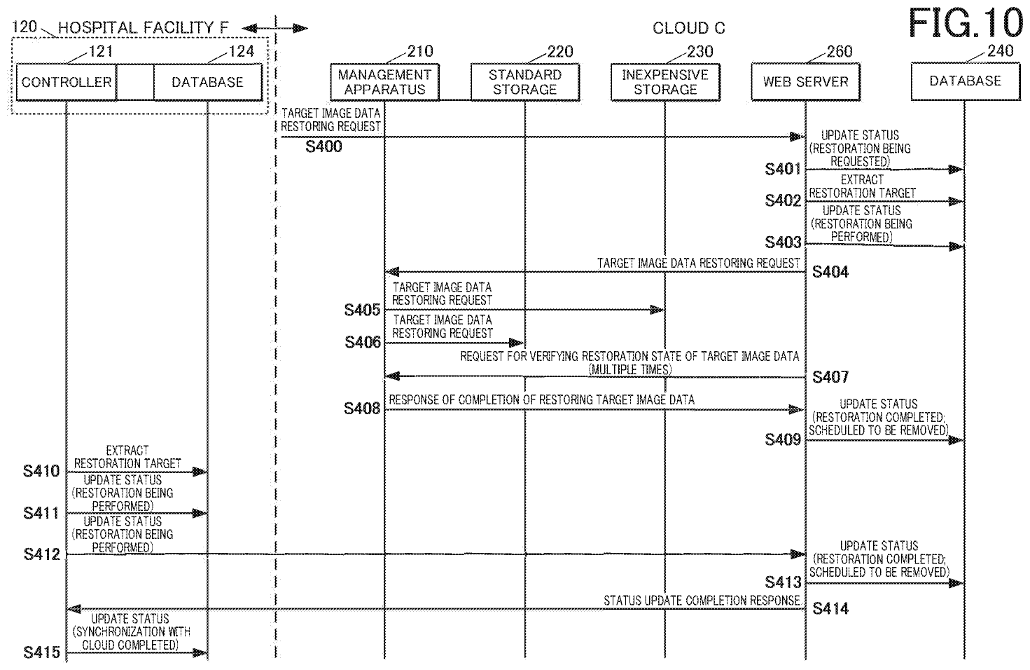

FIG. 10 is a sequence diagram of the medical information storing system 10 in a case of restoring the image data stored in the inexpensive storage 230 in the cloud C into the standard storage 220 in response to a reference request by the external user out of the hospital facility F. Note that according to a fourth sequence, the web server 260 that manages the database 240 and applies another action is provided in the cloud C. The information processing apparatus provided out of the hospital facility F may have the same components and functions as those of the information processing apparatus 150 shown in FIG. 5.

The web server 260 accepts a reference request for a predetermined image data, from an external information processing apparatus out of the hospital facility F (Step S400). The user can confirm and select the image data that they intend to refer to from the work list displayed on the display. In this case, as described above, to restore the target image data stored in the inexpensive storage 230 into the standard storage 220, the warning information indicating that a considerable time period is required for restoration, and the selection information for selecting whether to download or not, may be displayed for the user, as shown in FIG. 8.

Upon receipt of the restoration request for the target image data from the external information processing apparatus out of the hospital facility F, the web server 260 outputs, to the database 240, the update instruction for updating the status in the database 240 in the cloud C (Step S401). The web server 260 functions as a management device. The update instruction includes a status code “13: RESTORATION IN STORAGE BEING REQUESTED” indicating that the target image data stored in the inexpensive storage 230 are to be restored into the standard storage 220. Upon receipt of the update instruction from the web server 260, the database 240 updates the status code in the management table 240a to “13: RESTORATION IN STORAGE BEING REQUESTED”.

The web server 260 issues a verification request (polling) to the database 240, and extracts, from the database 240, restoration target information about the target image data for which the restoration request has been issued (Step S402).

The web server 260 outputs, to the database 240, an update instruction for updating the status of the target image data for which the restoration request has been issued (Step S403). The update instruction includes the status code “14: RESTORATION IN STORAGE BEING PERFORMED” indicating that the target image data are being restored from the inexpensive storage 230 into the standard storage 220. Upon receipt of the update instruction from the web server 260, the database 240 updates the status code of the target image data from “13: RESTORATION IN STORAGE BEING REQUESTED” to “14: RESTORATION IN STORAGE BEING PERFORMED”.

The web server 260 transmits a restoration request instruction for restoring the target image data from the inexpensive storage 230 into the standard storage 220, to the management apparatus 210 via the network N (Step S404). Here, an authentication key issuing service may be used as a condition for accessing the management apparatus 210 or the like in the cloud C.

If the management apparatus 210 receives the restoration request instruction from the web server 260, the management apparatus 210 outputs, to the inexpensive storage 230, a restoration instruction for restoring the target image data into the standard storage 220 (Step S405). Upon receipt of the moving instruction from the management apparatus 210, the inexpensive storage 230 reads the target image data, and moves the data to the standard storage 220.

Likewise, the management apparatus 210 outputs a restoration instruction for restoring the target image data, to the standard storage 220 (Step S406). Upon receipt of the restoration instruction from the management apparatus 210, the standard storage 220 stores the target image data read from the inexpensive storage 230. In one or more embodiments, after copying and storing of the target image data to and into the standard storage 220 is completed, the management apparatus 210 may automatically output the restored target image data to the information processing apparatus 150 having issued the reference request. For example, after the target image data are output, the management apparatus 210 may remove the target image data stored in the standard storage 220 in accordance with a period set by the user. In this case, the scheduled removal time at which the target image data are removed from the standard storage 220 may be displayed on the display 154 of the information processing apparatus 150.

The web server 260 transmits a verification instruction for verifying the restoration state of the target image data to the management apparatus 210 via the network N (Step S407). The verification instruction is issued once and then finished in some cases, and is issued multiple times and then finished in other cases. In this case, as shown in FIG. 7, the verification instruction described above may include a verification instruction for confirming scheduled removal of the target image data.

If the management apparatus 210 receives the verification instruction from the medical image management apparatus 120, the management apparatus 210 verifies whether restoration of the target image data from the inexpensive storage 230 to the standard storage 220 has been completed, or is being performed. If the target image data have been restored, the management apparatus 210 transmits a response of completion of restoring the target image data, to the web server 260 via the network N (Step S408).

If the web server 260 receives the response of completion of restoring the target image data from the management apparatus 210, the web server 260 outputs, to the database 240, the update instruction for updating the status of the target image data in the database 240 in the cloud C (Step S409). The web server 260 functions as a management device and an output device. The update instruction includes a status code “20: RESTORATION IN STORAGE COMPLETED” indicating that the restoration of the target image data from the inexpensive storage 230 into the standard storage 220 has been completed. Upon receipt of the update instruction from the management apparatus 210, the database 240 updates the status in the management table 240a from “14: RESTORATION IN STORAGE BEING PERFORMED” to “20: RESTORATION IN STORAGE COMPLETED”. At this stage, synchronization with the database 124 on the hospital facility F side is not made.

For example, also in the hospital facility F, the reference request for the target image data described above is received in some cases. In such cases, the controller 121 of the medical image management apparatus 120 extracts, from the database 124, restoration target information, such as a status about the target image data (Step S410).

The controller 121 transmits, to the database 124, an update instruction for updating the status of the target image data in the database 124 (Step S411). Upon receipt of the update instruction from the controller 121, the database 124 updates the status code of the target image data to “13: RESTORATION IN STORAGE BEING REQUESTED”.

The controller 121 transmits an update instruction for updating the status of the target image data in the database 240 in the cloud C, to the web server 260 via the network N (Step S412). The update instruction includes the status code “14: RESTORATION IN STORAGE BEING PERFORMED” indicating that the target image data are being restored.

If the web server 260 receives the update instruction from the medical image management apparatus 120, the web server 260 verifies the status of the target image data in the database 240 in the cloud (Step S413). In one or more embodiments, at this stage, the target image data are restored from the inexpensive storage 230 into the standard storage 220.

The web server 260 transmits a response indicating that the status of the target image data in the database 240 is in the already restored state, to the medical image management apparatus 120 via the network N (Step S414).

Upon receipt of the response indicating the already restored state from the web server 260, the controller 121 of the medical image management apparatus 120 updates the status code of the target image data to “22: SYNCHRONIZATION BETWEEN STORAGE AND CLOUD COMPLETED” (Step S415). As described above, when the restoration request is issued about the identical target image data from the hospital facility F side, the status of the target image data in the database 124 on the hospital facility F side is synchronized with the status of the target image data in the database 240 on the cloud C side.

As described above, according to one or more embodiments, the functionalities of the system, especially control technique for storages having different performances are improved, and thus provide improvements in medical information management technology. Specifically, when a reference request for the image data stored in the inexpensive storage 230 in the cloud C is issued, the display 154 displays, on the work list, the status indicating the storing state of the image data, and the like. Accordingly, the user can correctly grasp the status of the target image data that they intend to refer to. For example, if the target image data are in the inexpensive storage 230, the user can confirm technical issues specific to the cloud C, such as a certain time period required for reading, and the accompanying cost, before downloading the target image data. As a result, the image data stored in the standard storage 220 and the inexpensive storage 230 in the cloud C can be efficiently managed.

According to one or more embodiments, for example, if the image data are in the inexpensive storage 230, warning indicating that restoration requires a certain time period, or the like is displayed. Consequently, at this stage, the user can determine whether to stop or continue reference to and restoration of the image data. Accordingly, in a case of focusing on the cost, working time period and the like, restoration of the image data is stopped, which can facilitate reduction in cost, and facilitate reduction in a working time period.

According to one or more embodiments, similar to the inside of the hospital facility F, also from the external cloud C side out of the hospital facility F, the status of the image data intended to be referred to can be verified on the screen of the display. Accordingly, the user can grasp the status of the image data intended to be referred to, the image data can be efficiently managed for the standard storage 220 and the inexpensive storage 230 in the cloud C. For example, if the image data are in the inexpensive storage 230, warning indicating that restoration requires a certain time period, or the like is displayed. Consequently, at this stage, it can be determined whether to stop or continue reference to and restoration of the image data.

According to one or more embodiments, by presetting a designated condition, image data that have not been referred to for a predetermined period, and image data having a low possibility of being referred to thereafter are moved by the management apparatus 210 from the standard storage 220 to the inexpensive storage 230. Accordingly, the image data can be more efficiently managed, and cost reduction can be facilitated at the same time.

The embodiments of this disclosure have thus been described in detail with reference to the accompanying drawings. However, the technical scope of this disclosure is not limited to such an example. A person having ordinary skill in the technical field of this disclosure understands that various modifications and improvements applied in the scope of the technical spirit described in the claims naturally belong to the technical scope of this disclosure.

In a case where the medical image data, and compressed data obtained by reducing a data size of the medical image data are available, the hardware processor transfers only the medical image data to the second storage. For example, if the medical image management apparatus 120 and the like include the image data, and compressed data obtained by reducing the data size of the image data, the compressed data may be stored in the standard storage 220, and only the image data may be moved to the inexpensive storage 230. The compressed data may be, for example, thumbnail image data. This allows efficient storage management. In a case where the image data are moved from the standard storage 220 to the inexpensive storage 230, the medical image management apparatus 120 and the like may integrate multiple pieces of image data into one file, and move the file to the inexpensive storage 230. This can facilitate improvement in efficiency of data transmission.

Also in a case where the image data stored in the inexpensive storage 230 are referred to from the external information processing apparatus or the like out of the hospital facility F, display control similar to that of the information processing apparatus 150 in the hospital facility F can be provided. Specifically, when a reference request for the image data is issued from the external information processing apparatus or the like out of the hospital facility F, the scheduled restoration time of the target image data to be restored in the standard storage 220 may be displayed as warning on the display screen. Further, when the image data are restored in the standard storage 220, a scheduled removal time at which the restored image data are removed from the standard storage 220 may be displayed on the display screen. This allows the user to grasp the restoration time period of the target image data, which can facilitate improvement in working efficiency.

After copying of the image data to the standard storage 220 is completed, the medical image management apparatus 120 or the like may perform control of automatically downloading the restored image data to the information processing apparatus 150 or the like having issued the reference request. Selection information for selecting whether to download the image data or not after the image data are restored may be displayed on the display 154 of the information processing apparatus 150.

Although embodiments of the present invention have been described and illustrated in detail, the disclosed embodiments are made for purposes of illustration and example only and not limitation. The scope of the present invention should be interpreted by terms of the appended claims.

What is claimed is:

1. A medical information storing system, comprising:
a hardware processor that:
stores medical data related to medical care in a first storage provided in a cloud;
transfers, from the first storage, the medical data satisfying a designated condition to a second storage provided in the cloud, wherein the second storage requires a longer reading time period than the first storage does;
manages a storing state of the medical data; and
outputs to a display the storing state, or warning information about restoration of the medical data stored in the second storage and having been referred to.

2. The medical information storing system according to claim 1, wherein
the designated condition is at least one of an examination date of a patient, and an examination receipt date.

3. The medical information storing system according to claim 1, wherein
the designated condition is a lapse of a predetermined period from a latest examination registration of a patient, or a lapse of a predetermined period from hospital visiting of the patient.

4. The medical information storing system according to claim 1, wherein
the designated condition is death of a patient, or complete recovery of the patient.

5. The medical information storing system according to claim 1, wherein
the hardware processor outputs to the display the warning information about the restoration of the medical data stored in the second storage and having been referred to.

6. The medical information storing system according to claim 1, further comprising:
a first database that is provided in a facility, and manages the storing state of the medical data; and
a second database that is provided in the cloud, and manages the storing state of the medical data, wherein
the hardware processor synchronizes the storing state of the medical data in the first database with the storing state of the medical data in the second database.

7. The medical information storing system according to claim 1, wherein
the hardware processor outputs the storing state or the warning information to an information processing apparatus provided in a facility, and to an information processing apparatus provided in the cloud.

8. The medical information storing system according to claim 1, wherein
once the medical data stored in the second storage are referred to, the hardware processor copies the medical data stored in the second storage to the first storage, and
the hardware processor removes the medical data copied to the first storage, in accordance with a period set by a user.

9. The medical information storing system according to claim 8, wherein
the hardware processor outputs a scheduled removal time at which the medical data copied to the first storage are removed from the first storage.

10. The medical information storing system according to claim 1, wherein
the medical data includes medical image data, and
in a case where the medical image data, and compressed data obtained by reducing a data size of the medical image data are available, the hardware processor transfers only the medical image data to the second storage.

11. The medical information storing system according to claim 1, wherein
when transferring the medical data to the second storage, the hardware processor integrates multiple pieces of the medical data into one file, and transfers the file to the second storage.

12. The medical information storing system according to claim 1, wherein
once the medical data stored in the second storage are referred to, the hardware processor outputs a scheduled restoration time of the medical data to be restored in the first storage.

13. The medical information storing system according to claim 1, wherein
once copying of the medical data to the first storage is completed, the hardware processor automatically outputs the medical data that have been restored to an external apparatus having transmitted a reference request.

14. The medical information storing system according to claim 1, wherein
once the medical data stored in the second storage are referred to, the hardware processor outputs selection information for selecting whether to download the medical data or not after the restoration of the medical data.

15. A medical information storing method, causing a hardware processor to execute:
storing medical data related to medical care in a first storage provided in a cloud;
transferring, from the first storage, the medical data satisfying a designated condition to a second storage provided in the cloud, wherein the second storage requires a longer reading time period than the first storage does;
managing a storing state of the medical data; and
outputting to a display the storing state, or warning information about restoration of the medical data stored in the second storage and having been referred to.

16. The medical information storing method according to claim 15, the method further causing the hardware processor to execute:
outputting to the display the warning information about the restoration of the medical data stored in the second storage and having been referred to.

17. A non-transitory computer readable recording medium storing instructions causing a computer to execute:
storing medical data related to medical care in a first storage provided in a cloud;
transferring, from the first storage, the medical data satisfying a designated condition to a second storage provided in the cloud, wherein the second storage requires a longer reading time period than the first storage does;
managing a storing state of the medical data; and
outputting to a display the storing state, or warning information about restoration of the medical data stored in the second storage and having been referred to.

18. The non-transitory computer readable recording medium according to claim 17, wherein the instructions cause the computer to execute:
outputting to the display the warning information about the restoration of the medical data stored in the second storage and having been referred to.
